(19) United States
(12) Patent Application Publication
(10) Pub. No.: US 2023/0225599 A1
(43) Pub. Date: Jul. 20, 2023

(54) METHODS AND DEVICES FOR MULTI-SPECTRAL IMAGING

(71) Applicants: Cilag GmbH International, Zug (CH); JENOPTIK Optical Systems GmbH, Jena (DE)

(72) Inventors: Robert Trusty, Cincinnati, OH (US); Jeremiah Henley, Fair Oaks, CA (US); Hannes Weise, Jena (DE); Ralf Hambach, Jena (DE)

(21) Appl. No.: 17/580,194

(22) Filed: Jan. 20, 2022

(51) Int. Cl. A61B 1/05; G02B 23/24; G01J 3/28

(52) U.S. Cl. CPC A61B 1/05; G01J 3/2823; G02B 23/243; G01J 2003/2826

(57) ABSTRACT

An imaging system includes a first optical system configured to receive an imaging beam from a surgical region. The imaging beam including a first wavelength band and a second wavelength band. The imaging beam is directed along a first optical axis. The first optical system includes a dichroic beam splitter, and the first optical system is configured to direct a first optical beam associated with the first wavelength band along a first direction and direct a second optical beam associated with the second wavelength band along a second direction. The imaging system also includes a first sensor located along the first direction and configured to capture a first image associated with the first optical beam. The image system further includes a first relay lens system located along the second direction downstream from the first optical system and configured to receive the second optical beam.

### FIELD

The present disclosure relates generally to multi-spectral imaging for tissue visualization during surgery.

### BACKGROUND

Surgical systems often incorporate an imaging system, which can allow the clinician(s) to view the surgical site and/or one or more portions thereof on one or more displays such as a monitor. The display(s) can be local and/or remote to a surgical theater. An imaging system can include a scope with a camera or sensor that views the surgical site and transmits the view to a display that is viewable by a clinician. Scopes include, but are not limited to, laparoscopes, arthroscopes, angioscopes, bronchoscopes, choledochoscopes, colonoscopes, cytoscopes, duodenoscopes, enteroscopes, esophagogastro-duodenoscopes (gastroscopes), endoscopes, laryngoscopes, nasopharyngo-neproscopes, sigmoidoscopes, thoracoscopes, ureteroscopes, and exoscopes.

By way of example, certain concealed structures, physical contours, and/or dimensions of structures within a surgical field may be unrecognizable intraoperatively by certain imaging systems. Additionally, certain imaging systems may be incapable of communicating and/or conveying certain information regarding the concealed structures to clinician(s) intraoperatively.

Accordingly, there remains a need for improved imaging techniques for tissue visualization during surgery.

### SUMMARY

Various aspects of the disclosed subject matter may provide one or more of the following capabilities.

In an aspect, an imaging system includes a first optical system configured to receive an imaging beam from a surgical region. The imaging beam including a first wavelength band and a second wavelength band. The imaging beam is directed along a first optical axis. The first optical system includes a dichroic beam splitter, and the first optical system is configured to direct a first optical beam associated with the first wavelength band along a first direction and direct a second optical beam associated with the second wavelength band along a second direction. The imaging system also includes a first sensor located along the first direction and configured to capture a first image associated with the first optical beam. The image system further includes a first relay lens system located along the second direction downstream from the first optical system and configured to receive the second optical beam at a first end of the first relay lens system and transmit at least a portion of the second optical beam via a second end of the first relay lens system. The imaging system also includes a second sensor located downstream from the first relay lens system and adjacent to the second end of the first relay lens system. The second sensor is configured to capture a second image associated with the second optical beam.

One or more of the following features can be included in any feasible combination.

In some implementations, the first optical system, the first sensor, the first relay lens system and the second sensor are located at a distal end of a surgical scope device. In some implementations, the surgical scope device is configured to receive the imaging beam in the surgical region and guide the imaging beam to the first optical system. In some implementations, the surgical scope device is a stereo scope. In some implementations, the surgical scope device is one of an endoscope and a laparoscope. In some implementations, at least one optical element in the first optical system is a 45 degree prism. The pentaprism includes the dichroic beam splitter.

In some implementations, at least one optical element in the first optical system is a pentaprism, The pentaprism includes the dichroic beam splitter. In some implementations, the dichroic beam splitter is located at a proximal surface of the pentaprism. In some implementations, the first sensor is located at a first image plane and the second sensor is located at a second image plane. A first distance of the first sensor relative to the first optical system is less than a second distance of the second sensor relative to the first optical system. In some implementations, a first size of the first image detected by the first sensor is different from a second size of a second image detected by the second sensor.

In some implementations, an active optical area of the first sensor and an active area of the second sensor are of different sizes. In some implementations, the first direction is perpendicular to the second direction. In some implementations, a light source is used to illuminate the object to be imaged. In some implementations, the light source includes a plurality of individually selectable narrow or wide wavelength bands. In some implementations, the light source includes one or more of lasers, light emitting diodes and incandescent sources configured to generate the narrow or wide wavelength bands.

In some implementations, the imaging system further includes a second optical system configured to receive the imaging beam from the surgical region. The second optical system is configured to direct a third optical beam associated with the first wavelength band along a third direction and direct a fourth optical beam associated with the second wavelength band along a fourth direction. The imaging system further includes a third sensor located along the third direction and configured to capture a third image associated with the third optical beam. The imaging system further incudes a second relay lens system located along the fourth direction downstream from the second optical system and configured to receive the fourth optical beam at a first end of the second relay lens system and transmit at least a portion of the fourth optical beam via a second end of the second relay lens system. The imaging system also includes a fourth sensor located downstream from the second relay lens system and adjacent to the second end of the second relay lens system. The fourth sensor is configured to capture a second image associated with the second optical beam.

In an aspect, an imaging system includes a first optical system configured to receive an imaging beam from a surgical region, the imaging beam including a first wavelength band and a second wavelength band, wherein the imaging beam is directed along a first optical axis. The first optical system includes a dichroic beam splitter. The first optical system is configured to direct a first optical beam associated with the first wavelength band along a first direction and direct a second optical beam associated with the second wavelength band along a second direction. The imaging system further includes a first sensor located along the first direction and configured to capture a first image associated with the first optical beam. The imaging system further includes a second sensor located along the second direction downstream from the first optical module and configured to receive the second optical beam. The second sensor is configured to capture a second image associated with the second optical beam.

In an aspect, a surgical instrument includes a surgical scope device including a distal end and a proximal end. The distal end of the surgical scope device is configured to be placed in a surgical region. The surgical instrument further includes an imaging system located in the distal end of the surgical scope device. The imaging system includes a first optical system configured to receive an imaging beam from a surgical region. The imaging beam including a first wavelength band and a second wavelength band. The imaging beam is directed along a first optical axis. The first optical system includes a dichroic beam splitter, and the first optical system is configured to direct a first optical beam associated with the first wavelength band along a first direction and direct a second optical beam associated with the second wavelength band along a second direction. The imaging system also includes a first sensor located along the first direction and configured to capture a first image associated with the first optical beam. The image system further includes a first relay lens system located along the second direction downstream from the first optical system and configured to receive the second optical beam at a first end of the first relay lens system and transmit at least a portion of the second optical beam via a second end of the first relay lens system. The imaging system also includes a second sensor located downstream from the first relay lens system and adjacent to the second end of the first relay lens system. The second sensor is configured to capture a second image associated with the second optical beam.

In some implementations, the proximal end of the surgical scope includes a processor configured to receive a first signal representative of the first image detected by the first sensor and receive a second signal representative of the second image detected by the second sensor. In some implementations, the processor is configured to generate a modified image that includes a superposition of at least a portion of the first image and at least a portion of the second image.

In an aspect, a method includes receiving, via a first optical system, an imaging beam from a surgical region, wherein the imaging beam includes a first wavelength band and a second wavelength band, and is directed along a first optical axis. The method also includes directing, by a dichroic beam splitter, a first optical beam associated with the first wavelength band along a first direction and directing a second optical beam associated with the second wavelength band along a second direction. The method further includes capturing a first image associated with the first optical beam. The first image is captured by a first sensor located along the first direction. The method further includes receiving the second optical beam by a first relay system located along the second direction downstream from the first optical system. The second optical beam is received at a first end of the first relay lens system, and transmitting at least a portion of the second optical beam via a second end of the first relay lens system. The method further includes capturing a second image associated with the second optical beam. The second image is captured by a second sensor located along the second direction downstream from the first relay lens system and adjacent to the second end of the first relay lens system. In some implementations, the method further includes generating a modified image by at least superposing at least a portion of the first image and at least a portion of the second image.

Non-transitory computer program products (i.e., physically embodied computer program products) are also described that store instructions, which when executed by one or more data processors of one or more computing systems, causes at least one data processor to perform operations herein. Similarly, computer systems are also described that may include one or more data processors and memory coupled to the one or more data processors. The memory may temporarily or permanently store instructions that cause at least one processor to perform one or more of the operations described herein. In addition, methods can be implemented by one or more data processors either within a single computing system or distributed among two or more computing systems. Such computing systems can be connected and can exchange data and/or commands or other instructions or the like via one or more connections, including a connection over a network (e.g. the Internet, a wireless wide area network, a local area network, a wide area network, a wired network, or the like), via a direct connection between one or more of the multiple computing systems, etc.

### BRIEF DESCRIPTION OF DRAWINGS

This invention will be more fully understood from the following detailed description taken in conjunction with the accompanying drawings, in which:

FIG. 1 illustrates an exemplary multi-spectral surgical imaging system;

FIG. 2 illustrates an exemplary embodiment of a surgical instrument and a two-channel imaging system in the multi-spectral surgical imaging system of FIG. 1;

FIG. 3 illustrates an exemplary channel of the imaging system in FIG. 2;

FIG. 4 illustrates another exemplary channel of the imaging system in FIG. 2;

FIG. 5 illustrates an exemplary optical system of the imaging system in FIG. 2;

FIG. 6 illustrates another exemplary optical system of the imaging system in FIG. 2;

FIG. 7 illustrates an exemplary two-channel imaging system;

FIG. 8 is a schematic illustration of an exemplary control system of the multi-spectral surgical imaging system in FIG. 1; and

FIG. 9 illustrates a flowchart of an exemplary multi-spectral surgical imaging method.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Sizes and shapes of the systems and devices, and the components thereof, can depend at least on the anatomy of the subject in which the systems and devices will be used, the size and shape of components with which the systems and devices will be used, and the methods and procedures in which the systems and devices will be used.

The figures provided herein are not necessarily to scale. Further, to the extent arrows are used to describe a direction a component can be tensioned or pulled, these arrows are illustrative and in no way limit the direction the respective component can be tensioned or pulled. A person skilled in the art will recognize other ways and directions for creating the desired tension or movement. Likewise, while in some embodiments movement of one component is described with respect to another, a person skilled in the art will recognize that other movements are possible. Additionally, although terms such as “first” and “second” are used to describe various aspects of a component, e.g., a first end and a second end, such use is not indicative that one component comes before the other. Use of terms of this nature may be used to distinguish two similar components or features, and often such first and second components can be used interchangeably. Still further, a number of terms may be used throughout the disclosure interchangeably but will be understood by a person skilled in the art.

Multi-spectral imaging systems can be used in surgical procedures to acquire images or videos of a surgical region. For example, visible light can be used to image the surgical region, and radiations outside the visible light spectrum (e.g., infrared light) can be used to image portions of the surgical region that cannot be identified by visible light alone. The images associated with different wavelengths can be combined to generate a modified image of the surgical region that can be presented to a user (e.g., a surgeon) during surgery. In some implementations, surgical instruments (e.g., laparoscopes) can include a multi-spectral imaging system that can allow a surgeon to view the surgical region (e.g., multiple tissues in the surgical region) while performing the surgical procedure. For example, the multi-spectral imaging system can include imaging optics that can guide electromagnetic radiation in two or more wavelength bands from the surgical region (e.g., reflected by the surgical region) and an image sensor that can capture images associated with the electromagnetic radiation.

A single sensor may not be suitable for multi-spectral imaging due to the limited number of images that it can acquire in a given period of time (also referred to as framerate). Using multiple wavelength bands for imaging can reduce the number of frames of visible light images that can be captured in a given period. This can adversely affect the quality of the modified image displayed to the user. Therefore, it can be desirable to use multiple sensors to acquire images associated with the different wavelength bands. For example, visible light images can be captured by a first sensor and infrared images can be captured by a second sensor. Usage of multiple sensors can be achieved by using an optical system (e.g., one that includes one or more of dichroic beam splitters, prisms, pentaprism, and lenses) that can separate a first wavelength band (which can be directed to the first sensor) from a second wavelength band (which can be directed to the second sensor).

Typically, the first sensor and the second sensor are placed in a first image plane and a second image plane, respectively, of the optical system. The first and the second image planes can be located at similar distances from the optical system (e.g., adjacent to the optical system). As a result, in many known systems the first and the second sensors must have similar properties (e.g., generate images of similar sizes, resolution, framerates, etc.) to allow for synthesis of their respective images to generate the modified image. This is not desirable, as it can be challenging to find similar sensors that can also be placed in the image planes of the optical system. For example, if the optical element is located at the distal end of the surgical instrument, there may not be sufficient available space to fit two sensors in the image planes. In other known systems cameras that can capture images to be synthesized to generate a modified image can be customized to fit within the distal end of the surgical instrument. However, customized cameras can be unduly expensive and not easily available due to long lead times needed for manufacturing).

In some implementations of the system disclosed herein there is described a multi-spectral imaging system that can be configured to independently capture the images associated with the first wavelength band (e.g., visible light having wavelengths ranging from about 400 nanometers to about 800 nanometers) and the second wavelength band (e.g., infrared radiation having wavelengths ranging from about 800 nanometers to about 1000 nanometers). In some implementations, a relay lens system can be introduced in the optical path of the second wavelength band prior to imaging by the second sensor. As a result, the locations of the first image plane (where the first sensor is placed to capture the image associated with the first wavelength band) and the conjugate image plane (where the second sensor is placed to capture the image associated with the second wavelength band) can be independently adjusted. This allows for the design of a multi-spectral imaging system that can be efficiently placed in the distal end of a surgical instrument that may have limited space (e.g., placed within a region of small diameter at the distal end of the surgical instrument). Moreover, the independent determination of image planes allows for the usage of off-the-shelf sensors that can reduce the cost, complexity, and manufacturing time of the multi-spectral imaging system.

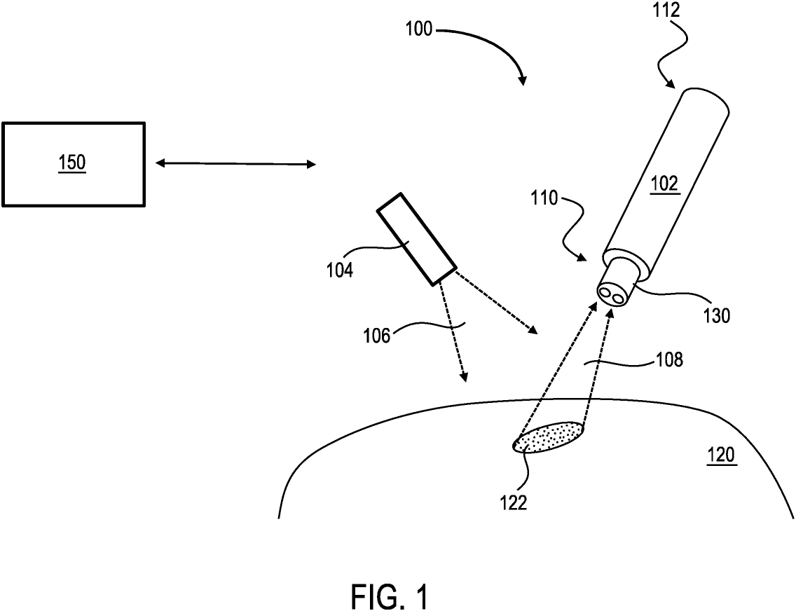

FIG. 1 illustrates an exemplary multi-spectral surgical imaging system 100 that includes a surgical instrument 102 capable of performing multi-spectral imaging. The surgical instrument 102 has a distal end 110 and a proximal end 112. The surgical instrument 102 can perform multi-spectral imaging of a portion 122 of a target tissue 120. In some implementations, the imaging system 100 can include a light source 104 that can illuminate the target tissue 120 with a beam 106, such as a multi-spectral beam. For example, the beam 106 can include radiation in multiple wavelength bands (e.g., visible light band, infrared band, etc.). In some implementations, the light source 104 can include one or more of sources of radiation (e.g., lasers, light emitting diodes, incandescent sources, etc.). For example, the light source 106 can include one or more of a first source that generates a first narrow wavelength band, a second source that generates a second narrow wavelength band, a third source that generates a first wide wavelength bands, a fourth source that generates a second wide wavelength band, etc.

The target tissue 120 (e.g., portion 122 of the target tissue 120) can interact with the beam 106 and generate an imaging beam 108, e.g., by reflecting a portion of the beam 106, generating new radiation based on interaction with the beam 106, etc. The imaging beam 108 can be captured by an imaging system 130 located at the distal end 110 of the surgical instrument 102. The multi-spectral surgical imaging system 100 can further include a control system 150 that can be operatively coupled to the surgical instrument 102 and the light source 104. The control system 150 can control aspects of the operation of the surgical instrument 102, by triggering image sensors in the surgical instrument 102 to capture images, process images captured by the image sensors, etc. The control system 150 can also control aspects of the light source 104, such as by triggering the visible light source and/or the infrared source in the light source 104 to generate the beam 106.

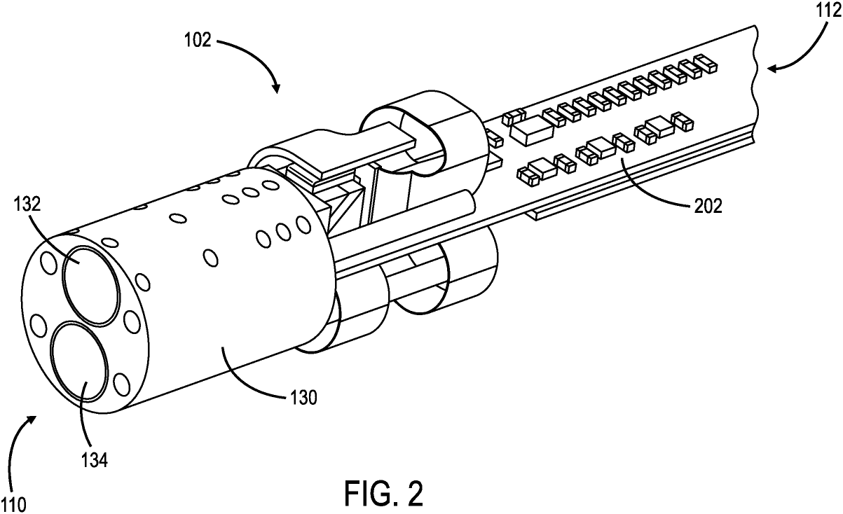

FIG. 2 illustrates an exemplary embodiment of the surgical instrument 102 that extends from the distal end 110 to the proximal end 112. The distal end 110 of the surgical instrument 102 can be introduced into a surgical environment that includes the target tissue 120 (not shown in FIG. 2). The surgical instrument 102 includes the imaging system 130 located at the distal end 110. The imaging system 130 can be configured to receive the imaging beam 108 from the target tissue 120 and capture images associated with the various wavelength bands included in the imaging beam 108 (e.g., which can correspond to the wavelength bands in the band 106 illuminated on the target tissue 120 by light source 104). The imaging system 130 can include multiple channels that can independently receive the imaging beam 108 and capture images associated with the various wavelength bands. For example, the imaging system 130 can include a first channel 132 and a second channel 134. A signal including data characterizing the captured images can be transmitted by each channel in the imaging system 130.

In some implementations, the signal(s) can be received and processed by processors 202 located at the proximal end of the surgical instrument 102. In some implementations, the signal(s) can be received and processed by the control system 150. It is desirable to capture the images at the distal end 110 and transmit a signal associated with the captured images to the proximal end instead of guiding the imaging beam 108 (or a portion thereof) through the surgical instrument 102 to the proximal end 112 and capturing the images at the proximal end. Capturing the images at the proximal end 112 guiding the imaging beam 108 through the surgical instrument 102 can be disadvantageous as it may require multiple optical elements (e.g., lenses, waveguides, etc.) that can lead to an increase in the cost and weight of the surgical instrument 102.

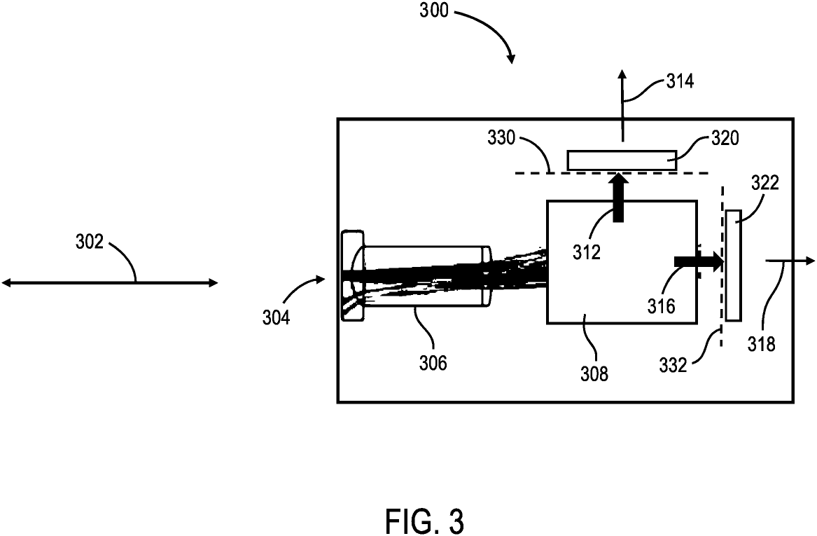

FIG. 3 illustrates an exemplary channel of an imaging system (e.g., imaging system 130) that can receive a multi-spectral imaging beam (e.g., imaging beam 108) from the surgical region and capture multiple images based on the wavelength band in the imaging beam. The imaging beam can be directed along an optical axis 302. In some implementations, the multi-spectral imaging beam can include a first wavelength band (e.g., including electromagnetic radiations in the visible light wavelength range) and a second wavelength band (e.g., including electromagnetic radiations in the infrared wavelength range).

The channel 300 can receive the imaging beam via an aperture 304, and a guiding optical system 306 can guide the imaging beam to an optical system 308 configured to separate different wavelength bands in the imaging beam. For example, the optical system 308 can guide a first optical beam 312 associated with the first wavelength band along a first direction 314, and guide a second optical beam 316 associated with the second wavelength band along the second direction 318. The optical system 308 can include a dichroic beam splitter (not shown) that can separate the first wavelength band from the second wavelength band. The guiding optical system 306 can include a telecentric objective that can provide constant magnification regardless of the distance of the target tissue from the surgical instrument 102.

In some implementations, the dichroic beam splitter (or dichroic filter) can separate shorter wavelengths from longer wavelengths relative to a predetermined wavelength (e.g., 650 nanometers). In some implementations, the dichroic filter can be designed as a shortpass filter where wavelengths longer than 650 nanometers are reflected and guided along the first direction 314, and wavelengths shorter than 650 nanometers are transmitted through and guided along the second direction 318. In some implementations, the dichroic filter can be designed as a longpass filter where wavelengths shorter than 650 nanometers are reflected and guided along a first direction 314, and wavelengths longer than 650 nanometers are transmitted and guided along the second direction 318. The dichroic filter can be designed to separate only a narrow band centered at a desired wavelength. The width of the separated band can be designed to accommodate tolerances and/or multiple desired wavelengths. The separated narrow band can be reflected or transmitted. The dichroic filter can include multiple separated narrow bands. The filter can include a combination of narrow bands and wide bands.

The channel 300 can include a first sensor 320 configured to capture an image of the surgical region associated with the first wavelength band, and a second sensor 322 configured to capture the image of the surgical region associated with the second wavelength band. The first sensor 320 is located at a first image plane 330 of the optical system 308, and the second sensor 322 is located at the second image plane 330 of the optical system 308. It can be desirable to place the sensors at their respective imaging planes in order to obtain sharp images of the surgical region. In some implementations, the first direction 314 and the second direction 318 can be perpendicular to each other (e.g., when the optical system 308 includes a 45 degrees prism). In other words, the first image plane 330 and the second image plane 332 can be perpendicular to each other. In some implementations, the first direction 314 and the second direction 318 can be at a non-perpendicular angle relative to each other. In other words, the first image plane 330 and the second image plane 332 can be oriented with respect to each other at a non-perpendicular angle. The image sensors 320 and 322 can have the same properties or different properties. In some implementations, the performance of an imaging system (e.g., imaging system 130) can be improved (e.g., optimized) by using sensors with different properties. For example, a first sensor can include a color filter array that can generate a color image from broadband white light; and a second sensor can be monochrome filter (e.g., includes no color filter array) to collect wavelengths of light outside the visible spectrum and/or wavelengths of interest for the multi-spectrum imaging system. In some implementations, the sensor collecting near-infrared light can have additional sensitivity to near-infrared light.

In some implementations, the location of the first image plane 330 and second image plane 332 relative to the optical system 308 is fixed based on the design of the optical system 308. For example, the first and the second image planes can be located close to the optical system 308 (e.g., at similar distances from the optical system 308) and the images formed at these planes can have similar properties (e.g., similar size, similar resolution, etc.). In such an implementation, it may be desirable that the first sensor 320 and the second sensor 322 have similar properties (e.g., generate images of similar sizes, resolution, etc.) to allow for synthesis of their respective images to generate the modified image.

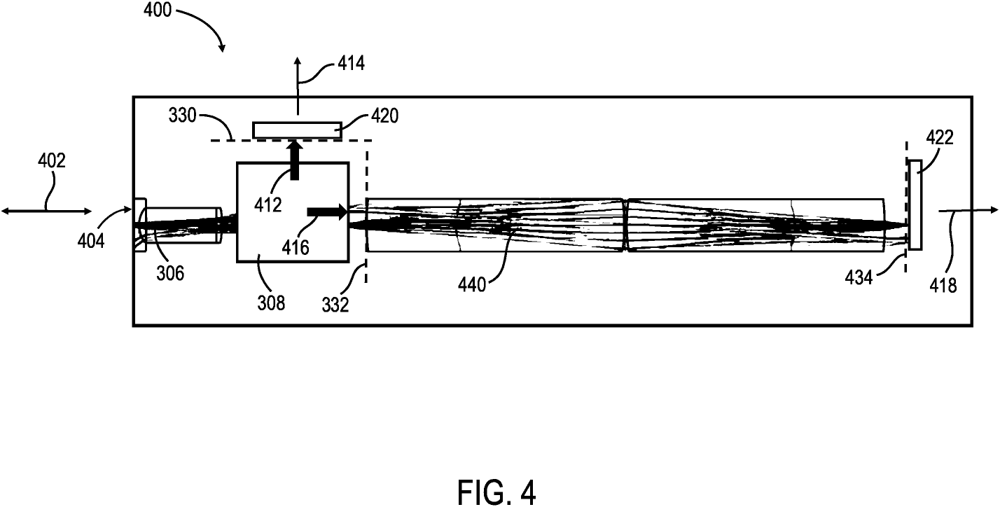

FIG. 4 illustrates an exemplary channel 400 of an imaging system (e.g., imaging system 130) that can perform multi-spectral imaging (e.g., of the imaging beam 108). As described below, the channel 400 includes a relay lens system that generate a conjugate image plane of an image plane in the channel 400. The imaging beam can be directed along an optical axis 402, and can be received by the channel 400 via the aperture 404. The guiding optical system 306 can guide the imaging beam to the optical system 308 that can guide a first optical beam 412 associated with the first wavelength band along a first direction 414, and guide a second optical beam 416 associated with the second wavelength band along the second direction 418.

The channel 400 can include a first sensor 420 configured to capture an image of the surgical region associated with the first wavelength band, and a second sensor 422 configured to capture the image of the surgical region associated with the second wavelength band. The first sensor 420 is located at the first image plane 330 of the optical system 308. The channel 400 includes a relay lens system 440 located along the second direction 418 downstream from the optical system 308. The relay lens system 440 can include a first end (located proximal to the second image plane 332) that can receive the second optical beam 416. The relay lens system 440 can include a second end via which the second beam 416 (or a portion thereof) can be transmitted.

One skilled in the art will understand that a relay lens system can include one or more lenses that can receive an image at one image plane and relay the image to another image plane. Relay lens systems can change the properties of the image from one image plane to another (e.g., vary the size / orientation of the image). The relay lens system 440 can generate a conjugate image plane 434 of the second image plane 332 of the optical system 308. In some implementations, the relay lens system 440 can generate a conjugate image at the conjugate image plane 434 that can have different properties from the image generated at the second image plane 332. For example, the relay lens system 440 can magnify the image at the second image plane 332 (e.g., the conjugate image can be larger than the image at the second image plane 332). Alternately, the relay lens system 440 can de-magnify the image at the second image plane 332 (e.g., the conjugate image can be smaller than the image at the second image plane 332).

In some implementations, the images formed at the first image plane 330 and the second image plane 332 can have similar properties (e.g., similar size, similar resolution, etc.). As described above, the conjugate image and the image formed at the second image plane 332 (and the image formed at the first image plane 330) can have different properties. This can allow the use of different sensors to capture the image at the first image plane 330 and the conjugate image at the conjugate image plane 434. In other words, the first sensor 420 and the second sensor 422 can have different properties. By way of example, the ability to use sensors with different properties can obviate the requirement of previously known systems to have two sensors that generate images of similar sizes and/or resolution, etc.

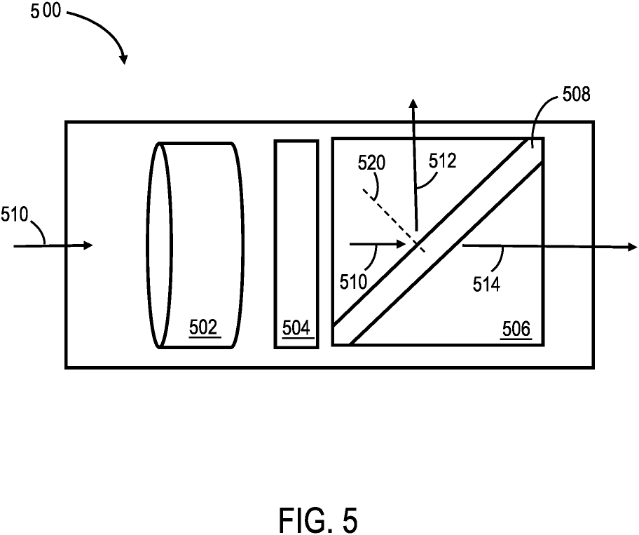

FIG. 5 illustrates an exemplary optical system 500 (e.g., optical system 308) configured to separate different wavelength bands in the imaging beam (e.g., imaging beam 108). The optical system 500 includes a lens 502, a notch filter 504 and a prism 506. The optical system 500 can receive the multi-spectral imaging beam 510 (e.g., from the surgical region via the guiding optical system 306), and separate the imaging beam 510 into a first optical beam 512 associated with the first wavelength band (e.g., including electromagnetic radiations in the visible light wavelength range) and a second optical beam 514 associated with the second wavelength band (e.g., including electromagnetic radiations in the infrared wavelength range).

In a typical arrangement the lens 502 can focus the imaging beam 510 while the notch filter 504 can block (or attenuate) a predetermined wavelength band while allowing other wavelength bands (e.g., visible light wavelength range, infrared wavelength range) that lie outside the predetermined wavelength band to pass through. The prism 506 can include a dichroic beam splitter 508 that can separate the first wavelength band from the second wavelength band in the multi-spectral imaging beam 510 (e.g., reflect the first optical beam 512 and transmit the second optical beam 514). In some implementations, the prism 506 can be about 45° prism, and the imaging beam 510 can be incident on the dichroic beam splitter 508 at a 45 degrees angle (e.g., angle between the imaging beam 510 and the normal 520 of the dichroic beam splitter 508 can be 45 degrees).

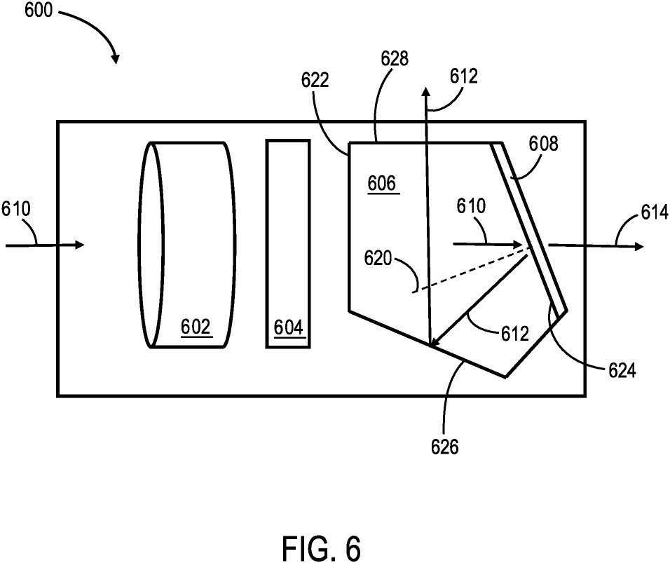

FIG. 6 illustrates another exemplary optical system 600 (e.g., optical system 308) configured to separate different wavelength bands in the imaging beam (e.g., imaging beam 108). The optical system 600 includes a lens 602, a notch filter 604 and a pentaprism 606. The optical system 600 can receive the multi-spectral imaging beam 610 (e.g., from the surgical region via the guiding optical system 306), and separate the imaging beam 610 into a first optical beam 612 associated with the first wavelength band (e.g., including electromagnetic radiations in the visible light wavelength range) and a second optical beam 614 associated with the second wavelength band (e.g., including electromagnetic radiations in the infrared wavelength range).

In a typical arrangement the lens 602 can focus the imaging beam 610 while the notch filter 604 can block (or attenuate) a predetermined wavelength band while allowing other wavelength bands (e.g., visible light wavelength range, infrared wavelength range) that lie outside the predetermined wavelength band to pass through. The pentaprism 606 can include a dichroic beam splitter 608 that can separate the first wavelength band from the second wavelength band in the multi-spectral imaging beam 610 (e.g., reflect the first optical beam 612 and transmit the second optical beam 614).

The imaging beam 610 can enter the pentaprism 606 through a first surface 622 (a distal surface) of the pentaprism 606. The dichroic beam splitter 608 located at a second surface 624 (a proximal surface) of the pentaprism 606 can transmit the second optical beam 614 and reflect the first optical beam 612. The first optical beam 612 is further reflected by the third surface 626 and emitted via the fourth surface 628 of the pentaprism 606. In some implementations, the imaging beam 610 can be incident on the dichroic beam splitter 608 at a 22.5 degrees angle (e.g., angle between the imaging beam 610 and the normal 620 of the dichroic beam splitter 608 can be 22.5 degrees). In some implementations, decreasing the angle of incidence (or the angle between the imaging beam and the normal of the dichroic beam splitter) can improve the separation between the first wavelength band and the second wavelength of the imaging beam. In other words, decreasing the angle of incidence can result in sharper edges of the transmission and reflection characteristics of the dichroic filter. This can reduce portions of the first wavelength band from being transmitted and/or portions of the second wavelength band from being reflected.

In some implementations, the orientation of the beam splitter 508 (or beam splitter 608) in the prism 506 (or prism 606) can vary. For example, the angle between the beam splitter 508 (or beam splitter 608) and a surface of the prism 506 (or prism 606) can vary. Based on the change in the angle of the beam splitter, the orientation of the sensors (e.g., first sensor 320, 420, second sensor 322, 422, etc.) can change. For example, the sensors in a given channel (e.g., first sensor 320 and second sensor 322 in channel 300, first sensor 420 and second sensor 422 in channel 400, etc.) may be oriented at a non-perpendicular angle.

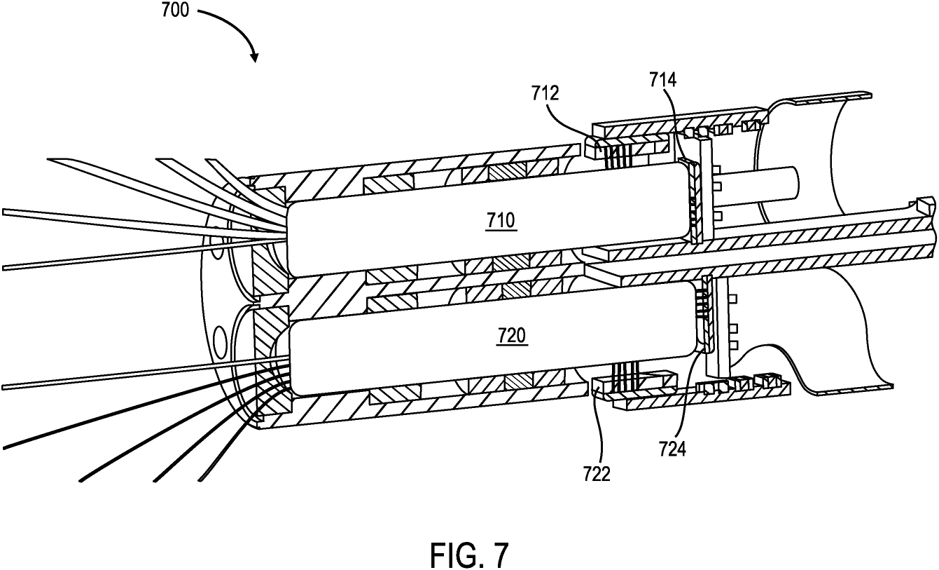

FIG. 7 illustrates an exemplary two-channel imaging system 700 that includes a first channel 710 (e.g., channel 300, channel 400, etc.) and a second channel 720 (e.g., channel 300, channel 400, etc.). The first channel 710 includes a first sensor 712 and a second sensor 714 that can capture the visible light image and infrared image of the surgical region, respectively. The first channel 720 includes a third sensor 722 and a fourth sensor 724 that can capture the visible light image and infrared image of the surgical region, respectively. The two-channel imaging system 700 can allow for simultaneous capture of four images (e.g., two visible light images and two infrared images) via four sensors (e.g., first sensor 712, second sensor 714, third sensor 722 and fourth sensor 724). This can improve the image throughput of the two-channel imaging system 700 in comparison to a single channel imaging system.

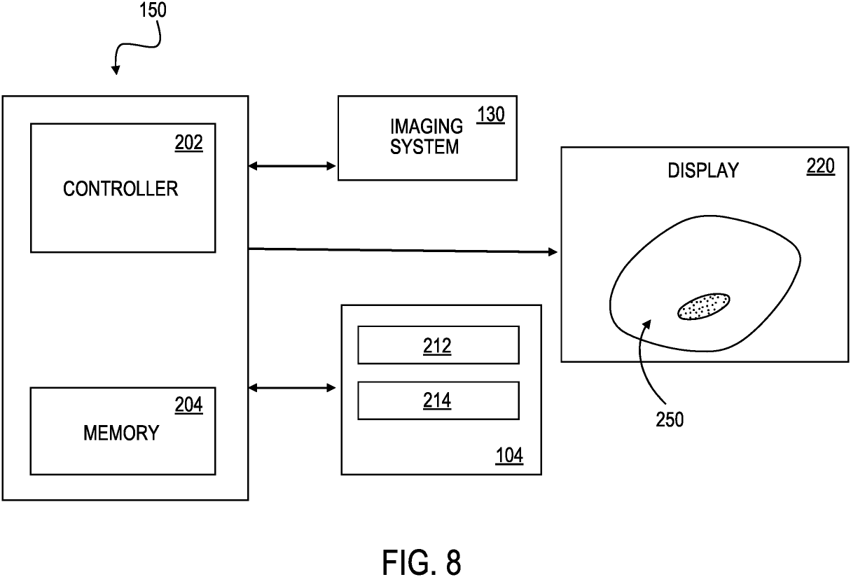

FIG. 8 is a schematic illustration of the exemplary control system 150 of multi-spectral surgical imaging system 100. As shown, the control system 150 includes a controller 202 having at least one processor that is in operable communication with, among other components, a memory 204, visible light radiation source 212 and infrared radiation source 214 (e.g., included in the source 104), the surgical instrument 102, and the display 220. The memory 204 is configured to store instructions executable by the processor of the controller 202 to process images captured by the image sensors in the imaging system 130 of the surgical instrument 102. For example, the controller 202 can generate a modified image 250 that includes a superposition of the first image (or a portion thereof) captured by the visible light sensor (e.g., first sensor 320, 420) and the second image (or a portion thereof) captured by the visible light sensor (e.g., second sensor 322, 422). The modified image can be displayed in the display 220.

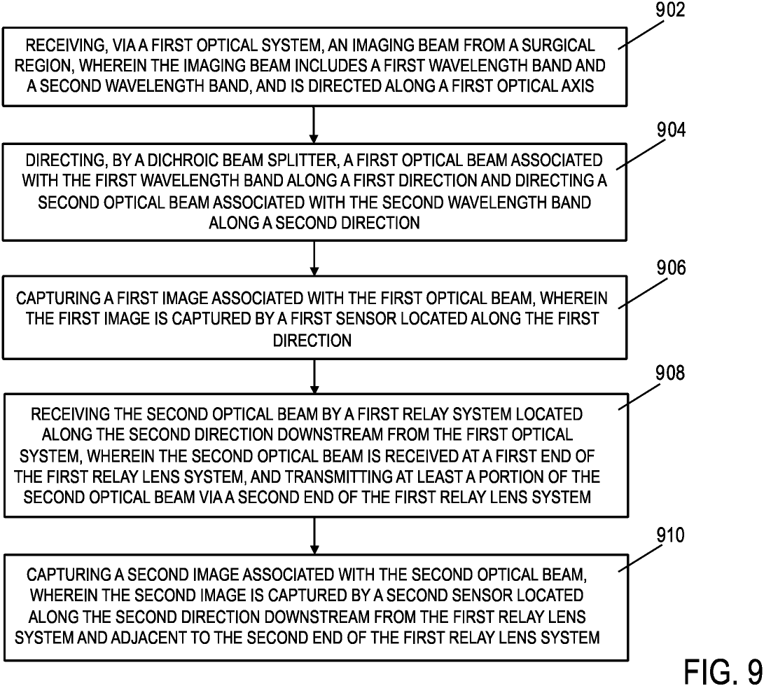

FIG. 9 illustrates a flowchart 900 of an exemplary multi-spectral surgical imaging method. At step 902, an imaging beam (e.g., imaging beam 108) can be received from a surgical region by an optical system (e.g., optical system 308) in an imaging system (e.g., imaging system 100). The imaging beam can include a first wavelength band and a second wavelength band, and is directed along an optical axis of the optical system. In some implementations, a guiding optical system (e.g., guiding optical system 306) can guide the imaging beam from an aperture of the imaging system 100 to the optical system. At step 904, a first optical beam associated with the first wavelength band can be directed along a first direction, and a second optical beam associated with the second wavelength band can be directed along a second direction by a dichroic beam splitter (e.g., dichroic beam splitter 508, dichroic beam splitter 608, etc.). At step 906, a first image associated with the first optical beam can be captured by a first sensor (e.g., first sensor 320, 420) located along the first direction. At step 908, the second optical beam can be received by a relay lens system (e.g. relay lens system 440) located along the second direction downstream from the optical system. The second optical beam can be received by a first end of the relay lens system. The second optical beam (or a portion thereof) can be transmitted via a second end of the first relay lens system. At step 910, a second image associated with the second optical beam is captured by a second sensor located along the second direction downstream from the relay lens system and adjacent to the second end of the relay lens system.

The multi-step imaging method can further include generating a modified image by superposing the first image (or a portion thereof) and the second image (or a portion thereof). The first image can be a visible light image of the surgical region, and the second image can be an infrared image of the surgical region. By superposing the visible light image and the infrared image to generate the modified image, information associated with both the visible light image and the infrared image can be simultaneously be presented (e.g., via a display to a surgeon). This can allow the surgeon to view portions of the surgical region that may not be visible by visible light image alone. Additionally, having separate sensors for capturing the visible light image and the infrared image can result in a high quality modified image of the surgical region (e.g., the modified image that can have a high frame rate).

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

In some implementations, source code can be human-readable code that can be written in program languages such as python, C++, etc. In some implementations, computer-executable codes can be machine-readable codes that can be generated by compiling one or more source codes. Computer-executable codes can be executed by operating systems (e.g., linux, windows, mac, etc.) of a computing device or distributed computing system. For example, computer-executable codes can include data needed to create runtime environment (e.g., binary machine code) that can be executed on the processors of the computing system or the distributed computing system.

Other embodiments are within the scope and spirit of the disclosed subject matter. For example, the prioritization method described in this application can be used in facilities that have complex machines with multiple operational parameters that need to be altered to change the performance of the machines. Usage of the word “optimize” / “optimizing” in this application can imply “improve” / “improving.”

Certain exemplary embodiments will now be described to provide an overall understanding of the principles of the structure, function, manufacture, and use of the systems, devices, and methods disclosed herein. One or more examples of these embodiments are illustrated in the accompanying drawings. Those skilled in the art will understand that the systems, devices, and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention. Further, in the present disclosure, like-named components of the embodiments generally have similar features, and thus within a particular embodiment each feature of each like-named component is not necessarily fully elaborated upon.

The subject matter described herein can be implemented in digital electronic circuitry, or in computer software, firmware, or hardware, including the structural means disclosed in this specification and structural equivalents thereof, or in combinations of them. The subject matter described herein can be implemented as one or more computer program products, such as one or more computer programs tangibly embodied in an information carrier (e.g., in a machine-readable storage device), or embodied in a propagated signal, for execution by, or to control the operation of, data processing apparatus (e.g., a programmable processor, a computer, or multiple computers). A computer program (also known as a program, software, software application, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file. A program can be stored in a portion of a file that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

The processes and logic flows described in this specification, including the method steps of the subject matter described herein, can be performed by one or more programmable processors executing one or more computer programs to perform functions of the subject matter described herein by operating on input data and generating output. The processes and logic flows can also be performed by, and apparatus of the subject matter described herein can be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processor of any kind of digital computer. Generally, a processor will receive instructions and data from a Read-Only Memory or a Random Access Memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, (e.g., EPROM, EEPROM, and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having a display device, e.g., a CRT (cathode ray tube) or LCD (liquid crystal display) monitor, for displaying information to the user and a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The techniques described herein can be implemented using one or more modules. As used herein, the term “module” refers to computing software, firmware, hardware, and/or various combinations thereof. At a minimum, however, modules are not to be interpreted as software that is not implemented on hardware, firmware, or recorded on a non-transitory processor readable recordable storage medium (i.e., modules are not software per se). Indeed “module” is to be interpreted to always include at least some physical, non-transitory hardware such as a part of a processor or computer. Two different modules can share the same physical hardware (e.g., two different modules can use the same processor and network interface). The modules described herein can be combined, integrated, separated, and/or duplicated to support various applications. Also, a function described herein as being performed at a particular module can be performed at one or more other modules and/or by one or more other devices instead of or in addition to the function performed at the particular module. Further, the modules can be implemented across multiple devices and/or other components local or remote to one another. Additionally, the modules can be moved from one device and added to another device, and/or can be included in both devices.

The subject matter described herein can be implemented in a computing system that includes a back-end component (e.g., a data server), a middleware component (e.g., an application server), or a front-end component (e.g., a client computer having a graphical user interface or a web interface through which a user can interact with an implementation of the subject matter described herein), or any combination of such back-end, middleware, and front-end components. The components of the system can be interconnected by any form or medium of digital data communication, e.g., a communication network. Examples of communication networks include a local area network (“LAN”) and a wide area network (“WAN”), e.g., the Internet.

Approximating language, as used herein throughout the specification and claims, may be applied to modify any quantitative representation that could permissibly vary without resulting in a change in the basic function to which it is related. Accordingly, a value modified by a term or terms, such as “about” and “substantially,” are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise.

What is claimed is:

1. An imaging system comprising:
a first optical system configured to receive an imaging beam from a surgical region, the imaging beam including a first wavelength band and a second wavelength band, wherein the imaging beam is directed along a first optical axis,
wherein the first optical system includes a dichroic beam splitter, the first optical system is configured to direct a first optical beam associated with the first wavelength band along a first direction and direct a second optical beam associated with the second wavelength band along a second direction;
a first sensor located along the first direction and configured to capture a first image associated with the first optical beam;
a first relay lens system located along the second direction downstream from the first optical system and configured to receive the second optical beam at a first end of the first relay lens system and transmit at least a portion of the second optical beam via a second end of the first relay lens system; and
a second sensor located downstream from the first relay lens system and adjacent to the second end of the first relay lens system, wherein the second sensor is configured to capture a second image associated with the second optical beam.

2. The imaging system of claim 1, wherein the first optical system, the first sensor, the first relay lens system and the second sensor are located at a distal end of a surgical scope device.

3. The imaging system of claim 2, wherein the surgical scope device is configured to receive the imaging beam in the surgical region and guide the imaging beam to the first optical system.

4. The imaging system of claim 2, wherein the surgical scope device is a stereo scope.

5. The imaging system of claim 2, wherein the surgical scope device is one of an endoscope and a laparoscope.

6. The imaging system of claim 1, wherein at least one optical element in the first optical system is a 45 degree prism, wherein the pentaprism includes the dichroic beam splitter.

7. The imaging system of claim 1, wherein at least one optical element in the first optical system is a pentaprism, wherein the pentaprism includes the dichroic beam splitter.

8. The imaging system of claim 7, wherein the dichroic beam splitter is located at a proximal surface of the pentaprism.

9. The imaging system of claim 1, wherein the first sensor is located at a first image plane and the second sensor is located at a second image plane, wherein a first distance of the first sensor relative to the first optical system is less than a second distance of the second sensor relative to the first optical system.

10. The imaging system of claim 1, wherein a first size of the first image detected by the first sensor is different from a second size of a second image detected by the second sensor.

11. The imaging system of claim 1, wherein an active optical area of the first sensor and an active area of the second sensor are of different sizes.

12. The imaging system of claim 1, wherein the first direction is perpendicular to the second direction.

13. The imaging system of claim 1, wherein a light source is used to illuminate the object to be imaged.

14. The imaging system of claim 13, wherein the light source includes a plurality of individually selectable narrow or wide wavelength bands.

15. The imaging system of claim 14, wherein the light source includes one or more of lasers, light emitting diodes and incandescent sources configured to generate the narrow or wide wavelength bands.

16. The imaging system of claim 1, further comprising:
a second optical system configured to receive the imaging beam from the surgical region, wherein the second optical system is configured to direct a third optical beam associated with the first wavelength band along a third direction and direct a fourth optical beam associated with the second wavelength band along a fourth direction;
a third sensor located along the third direction and configured to capture a third image associated with the third optical beam;
a second relay lens system located along the fourth direction downstream from the second optical system and configured to receive the fourth optical beam at a first end of the second relay lens system and transmit at least a portion of the fourth optical beam via a second end of the second relay lens system; and
a fourth sensor located downstream from the second relay lens system and adjacent to the second end of the second relay lens system, wherein the fourth sensor is configured to capture a second image associated with the second optical beam.

17. An imaging system comprising:
a first optical system configured to receive an imaging beam from a surgical region, the imaging beam including a first wavelength band and a second wavelength band, wherein the imaging beam is directed along a first optical axis,
wherein the first optical system includes a dichroic beam splitter, the first optical system is configured to direct a first optical beam associated with the first wavelength band along a first direction and direct a second optical beam associated with the second wavelength band along a second direction;
a first sensor located along the first direction and configured to capture a first image associated with the first optical beam;
a second sensor located along the second direction downstream from the first optical module and configured to receive the second optical beam, wherein the second sensor is configured to capture a second image associated with the second optical beam.

18. A surgical instrument comprising:
a surgical scope device including a distal end and a proximal end, wherein the distal end of the surgical scope device is configured to be placed in a surgical region; and
an imaging system located in the distal end of the surgical scope device, the imaging system including:
a first optical system configured to receive an imaging beam from a surgical region, the imaging beam including a first wavelength band and a second wavelength band, wherein the imaging beam is directed along a first optical axis
wherein the first optical system includes a dichroic beam splitter, the first optical system is configured to direct a first optical beam associated with the first wavelength band along a first direction and direct a second optical beam associated with the second wavelength band along a second direction;
a first sensor located along the first direction and configured to capture a first image associated with the first optical beam;
a first relay lens system located along the second direction downstream from the first optical system and configured to receive the second optical beam at a first end of the first relay lens system and transmit at least a portion of the second optical beam via a second end of the first relay lens system; and
a second sensor located downstream from the first relay lens system and adjacent to the second end of the first relay lens system, wherein the second sensor is configured to capture a second image associated with the second optical beam.

19. The surgical instrument of claim 17, wherein the proximal end of the surgical scope includes a processor configured to receive a first signal representative of the first image detected by the first sensor and receive a second signal representative of the second image detected by the second sensor.

20. The surgical instrument of claim 19, wherein the processor is configured to generate a modified image that includes a superposition of at least a portion of the first image and at least a portion of the second image.

21. A method comprising:
receiving, via a first optical system, an imaging beam from a surgical region, wherein the imaging beam includes a first wavelength band and a second wavelength band, and is directed along a first optical axis;
directing, by a dichroic beam splitter, a first optical beam associated with the first wavelength band along a first direction and directing a second optical beam associated with the second wavelength band along a second direction;
capturing a first image associated with the first optical beam, wherein the first image is captured by a first sensor located along the first direction;
receiving the second optical beam by a first relay system located along the second direction downstream from the first optical system, wherein the second optical beam is received at a first end of the first relay lens system, and transmitting at least a portion of the second optical beam via a second end of the first relay lens system; and
capturing a second image associated with the second optical beam, wherein the second image is captured by a second sensor located along the second direction downstream from the first relay lens system and adjacent to the second end of the first relay lens system.

22. The method of claim 21 further comprising generating a modified image by at least superposing at least a portion of the first image and at least a portion of the second image.
